SEQUENCE LISTING (73 sequences)

SEQ ID NO: 1 - DNA, 18 bp, Artificial sequence
  Synthetic primer
caacatggatgaaacctg

SEQ ID NO: 2 - DNA, 17 bp, Artificial sequence
  Synthetic primer
catcttcagcctgactg

SEQ ID NO: 3 - DNA, 17 bp, Artificial sequence
  Synthetic primer
tgcgcttcagcgcctaa

SEQ ID NO: 4 - DNA, 18 bp, Artificial sequence
  Synthetic primer
catggacgagatctcctc

SEQ ID NO: 5 - DNA, 20 bp, Artificial sequence
  Synthetic primer
aggctaaacccaatcagcat

SEQ ID NO: 6 - DNA, 17 bp, Artificial sequence
  Synthetic primer
cttctaggatgctagcg

SEQ ID NO: 7 - DNA, 17 bp, Artificial sequence
  Synthetic primer
tgctactgcagctctga

SEQ ID NO: 8 - DNA, 19 bp, Artificial sequence
  Synthetic primer
gaccaggaataacagtaag

SEQ ID NO: 9 - DNA, 17 bp, Artificial sequence
  Synthetic primer
tcagatgcattgggtgg

SEQ ID NO: 10 - DNA, 17 bp, Artificial sequence
  Synthetic primer
gaagttcagtggcttga

SEQ ID NO: 11 - DNA, 18 bp, Artificial sequence
  Synthetic primer
tctagaggcaggcattct

SEQ ID NO: 12 - DNA, 20 bp, Artificial sequence
  Synthetic primer
tcactgtctcaactgcctag

SEQ ID NO: 13 - DNA, 17 bp, Artificial sequence
  Synthetic primer
tactggcaggtctggat

SEQ ID NO: 14 - DNA, 20 bp, Artificial sequence
  Synthetic primer
atcctagattccaacttcct

SEQ ID NO: 15 - DNA, 17 bp, Artificial sequence
  Synthetic primer
tcagctgcaaacaggag

SEQ ID NO: 16 - DNA, 20 bp, Artificial sequence
  Synthetic primer
caccaagctcgatgaggctg

SEQ ID NO: 17 - DNA, 20 bp, Artificial sequence
  Synthetic primer
tgtagctcacctgcaccttg

SEQ ID NO: 18 - DNA, 20 bp, Artificial sequence
  Synthetic primer
aggaccaggtcgtgaatctg

SEQ ID NO: 19 - DNA, 20 bp, Artificial sequence
  Synthetic primer
gcagcaaagtccttgagtcc

SEQ ID NO: 20 - DNA, 20 bp, Artificial sequence
  Synthetic primer
ttcagacactatccctccgc

SEQ ID NO: 21 - DNA, 18 bp, Artificial sequence
  Synthetic primer
ctctgaggcctatggaga

SEQ ID NO: 22 - DNA, 17 bp, Artificial sequence
  Synthetic primer
cacagcggtgttcaacc

SEQ ID NO: 23 - DNA, 17 bp, Artificial sequence
  Synthetic primer
agacatcagcctccaca

SEQ ID NO: 24 - DNA, 18 bp, Artificial sequence
  Synthetic primer
gtatctccattgtaacgt

SEQ ID NO: 25 - DNA, 20 bp, Artificial sequence
  Synthetic primer
gaaggagctggagcaggtgt

SEQ ID NO: 26 - DNA, 17 bp, Artificial sequence
  Synthetic primer
agggtctgcataggttg

SEQ ID NO: 27 - DNA, 17 bp, Artificial sequence
  Synthetic primer
aagtacgccagtgagca

SEQ ID NO: 28 - DNA, 17 bp, Artificial sequence
  Synthetic primer
tggtgtgcgcctgtaat

SEQ ID NO: 29 - DNA, 19 bp, Artificial sequence
  Synthetic primer
ctgtagtactgtggactct

SEQ ID NO: 30 - DNA, 17 bp, Artificial sequence
  Synthetic primer
gagaatgcagtgccacc

SEQ ID NO: 31 - DNA, 17 bp, Artificial sequence
  Synthetic primer
aaccgagaatcactcct

SEQ ID NO: 32 - DNA, 17 bp, Artificial sequence
  Synthetic primer
ccatggtcttgagtccg

SEQ ID NO: 33 - DNA, 20 bp, Artificial sequence
  Synthetic primer
aaatggccagggttctcttt

SEQ ID NO: 34 - DNA, 17 bp, Artificial sequence
  Synthetic primer
actctgagctctgatcc

SEQ ID NO: 35 - DNA, 20 bp, Artificial sequence
  Synthetic primer
atagcactgtagactctgac

SEQ ID NO: 36 - DNA, 17 bp, Artificial sequence
  Synthetic primer
cgccatggcaatgaggc

SEQ ID NO: 37 - DNA, 20 bp, Artificial sequence
  Synthetic primer
cagggtcaccatcttgttaa

SEQ ID NO: 38 - DNA, 20 bp, Artificial sequence
  Synthetic primer
tctcaggcagactaggccat

SEQ ID NO: 39 - DNA, 20 bp, Artificial sequence
  Synthetic primer
tccgaaggactgagctcttg

SEQ ID NO: 40 - DNA, 20 bp, Artificial sequence
  Synthetic primer
tctcaggcagactaggccat

SEQ ID NO: 41 - DNA, 20 bp, Artificial sequence
  Synthetic primer
gatgactgccctgatcaagc

SEQ ID NO: 42 - DNA, 37 bp, Artificial sequence
  Synthetic primer
tgtaaaacgacggccagtagagcgaacctgtgcggct

SEQ ID NO: 43 - DNA, 34 bp, Artificial sequence
  Synthetic primer
tgtaaaacgacggccagtcgcagaacaccgtgtt

SEQ ID NO: 44 - DNA, 38 bp, Artificial sequence
  Synthetic primer
tgtaaaacgacggccagttgatcaccgcgttggtcacc

SEQ ID NO: 45 - DNA, 36 bp, Artificial sequence
  Synthetic primer
tgtaaaacgacggccagtaggctctgcgcgacgcca

SEQ ID NO: 46 - DNA, 37 bp, Artificial sequence
  Synthetic primer
tgtaaaacgacggccagtcagatcgaggtgaccttcg

SEQ ID NO: 47 - DNA, 36 bp, Artificial sequence
  Synthetic primer
tgtaaaacgacggccagtcatgaagagcgccgtgga

SEQ ID NO: 48 - DNA, 18 bp, Artificial sequence
  Synthetic primer
gacgggatccgcgacaag

SEQ ID NO: 49 - DNA, 20 bp, Artificial sequence
  Synthetic primer
ggtagtagtcgcctgacatg

SEQ ID NO: 50 - DNA, 18 bp, Artificial sequence
  Synthetic primer
cacggccacggacaagag

SEQ ID NO: 51 - DNA, 19 bp, Artificial sequence
  Synthetic primer
gaggactttgacaacaggc

SEQ ID NO: 52 - DNA, 18 bp, Artificial sequence
  Synthetic primer
tcagccaagaacgccctg

SEQ ID NO: 53 - DNA, 18 bp, Artificial sequence
  Synthetic primer
aggtcgatgccgatcgcc

SEQ ID NO: 54 - DNA, 19 bp, Artificial sequence
  Synthetic primer
gtcacgaagtacaggctgt

SEQ ID NO: 55 - DNA, 20 bp, Artificial sequence
  Synthetic primer
gtgcagtcggacatgaagca

SEQ ID NO: 56 - DNA, 19 bp, Artificial sequence
  Synthetic primer
gtaggccacagcctcgtcg

SEQ ID NO: 57 - DNA, 19 bp, Artificial sequence
  Synthetic primer
gctgacaccctctcgcgct

SEQ ID NO: 58 - DNA, 20 bp, Artificial sequence
  Synthetic primer
ctcgtccttctcgggccaag

SEQ ID NO: 59 - DNA, 20 bp, Artificial sequence
  Synthetic primer
tcgtctttcgagagtgactc

SEQ ID NO: 60 - DNA, 18 bp, Artificial sequence
  Synthetic primer
gattaggtggaggagtta

SEQ ID NO: 61 - DNA, 19 bp, Artificial sequence
  Synthetic primer
tctcgcgctgcacctcgtc

SEQ ID NO: 62 - DNA, 19 bp, Artificial sequence
  Synthetic primer
gagtcgatctccaggctgg

SEQ ID NO: 63 - DNA, 17 bp, Artificial sequence
  Synthetic primer
ctcgtccttctcggcca

SEQ ID NO: 64 - DNA, 15 bp, Artificial sequence
  Synthetic primer
gctctctgtcggctc

SEQ ID NO: 65 - DNA, 13 bp, Artificial sequence
  Synthetic primer
ttgacgcgaagcg

SEQ ID NO: 66 - DNA, 16 bp, Artificial sequence
  Synthetic primer
ggggtagaatgccttg

SEQ ID NO: 67 - DNA, 16 bp, Artificial sequence
  Synthetic primer
gcccgttgaagaagtc

SEQ ID NO: 68 - DNA, 17 bp, Artificial sequence
  Synthetic primer
gttggtgatggtgatct

SEQ ID NO: 69 - DNA, 15 bp, Artificial sequence
  Synthetic primer
caagatcagcgaggc

SEQ ID NO: 70 - DNA, 15 bp, Artificial sequence
  Synthetic primer
cgccggacactcaac

SEQ ID NO: 71 - DNA, 13 bp, Artificial sequence
  Synthetic primer
ggcggcccttgtc

SEQ ID NO: 72 - DNA, 16 bp, Artificial sequence
  Synthetic primer
ggctggtgaaccactt

SEQ ID NO: 73 - DNA, 14 bp, Artificial sequence
  Synthetic primer
aacgccctggagtc

(19) United States
(12) Patent Application Publication
(10) Pub. No.: US 2006/0046267 A1
(43) Pub. Date: Mar. 2, 2006

(54) Method for identification and determination of hypersensitivity of a patient to abacavir

(75) Inventor: Simon Mallal, Wembley (AU)

Correspondence Address: MARSHALL, GERSTEIN & BORUN LLP, 233 S. WACKER DRIVE, SUITE 6300, SEARS TOWER, CHICAGO, IL, 60606, US

(73) Assignee: Epipop Pty Ltd., West Perth (AU)

(21) Appl. No.: 10/504,047

(22) PCT Filed: Feb. 12, 2003
(86) PCT No.: PCT/AU03/00183

§ 371 Date: Jul. 13, 2005

(30) Foreign Application Priority Data
Feb. 12, 2002 (AU) PS0464

(51) Int. Cl. G01N 33/53

(52) U.S. Cl. 435/7.1

(57) ABSTRACT

A method for identifying whether a patient will exhibit a hypersensitive reaction or like-reaction to treatment with abacavir comprising the step of: typing the patient for the presence of the 57.1 ancestral haplotype.

### FIELD OF THE INVENTION

The present invention relates generally to the field of identification of patients who are susceptible to hypersensitivity to the HIV Reverse Transcriptase Inhibitor, abacavir. In particular, the invention provides method(s) for detecting patients who should avoid the use of abacavir because they are in a high risk category for abacavir hypersensitivity.

### BACKGROUND ART

Human immunodeficiency virus (HIV) is the etiological agent of a complex disease that includes progressive destruction of the immune system (acquired immune deficiency syndrome; AIDS) and degeneration of the central and peripheral nervous system. A common feature of HIV replication is the extensive post-translational processing of precursor polyproteins by a virally encoded protease to generate mature viral proteins required for virus assembly and function. Inhibition of this processing prevents the production of normally infectious virus.

It is known that some antiviral compounds which act as inhibitors of HIV replication are effective agents in the treatment of AIDS and similar diseases. Unfortunately however, there are some individuals who are hypersensitive to some of these inhibitors. Treatment with an inhibitor to which a person is hypersensitive may lead to a range of ailments and in rare instances, death. One such inhibitor that is particularly effective against HIV and which has these draw backs is abacavir.

Abacavir is a member of the nucleoside reverse transcriptase inhibitor (NRTI) class of antiretroviral drugs. It has a favourable profile in relation to the risk of mitochondrial toxicity, which is the major duration-dependent toxicity associated with NRTI use. It is commonly incorporated into co-formulation with zidovudine and lamivudine. These ‘triple NRTI’ regimens have demonstrated durable and potent antiviral activity that is comparable with regimens that combine antiretroviral drug classes.

Approximately 5-9% of patients treated with abacavir develop a hypersensitivity reaction. Symptoms usually appear within the first six weeks of treatment (median time to onset 11 days) and characteristically include fever, rash, gastrointestinal symptoms (nausea, vomiting, diarrhoea or abdominal pain) and lethargy or malaise. Less common manifestations include respiratory or musculoskeletal symptoms, headache, paresthesia, edema, renal or hepatic failure or anaphylaxis. Symptoms related to the hypersensitivity reaction worsen with continued therapy and usually improve within 24 hours of abacavir discontinuation. Rechallenging with abacavir following a hypersensitivity reaction typically results in the recurrence of symptoms within hours, and has the potential to induce a more severe clinical syndrome with increased risk of life-threatening hypotension and death.

In this context, a highly predictive test for abacavir hypersensitivity would provide a significant reduction in the total burden of toxicity associated with abacavir therapy, and allow for the safer use of this drug without inappropriate denial of access to its use.

The present invention seeks to present a method for detecting patients that are at risk of developing abacavir hypersensitivity.

### SUMMARY OF THE INVENTION

According to one embodiment, the invention provides a method for identifying whether a patient will exhibit a hypersensitive reaction or like-reaction to treatment with abacavir, comprising the step of: typing the patient for the presence of the 57.1 ancestral haplotype of the Major Histocompatibility Complex. Preferably, the ancestral haplotype is defined by the HLA-B*5701, C4A6, HLA-DR7, HLA-DQ3. In accordance with the present invention, patients that have the 57.1 ancestral haplotype are in a high risk category of developing a hypersensitive reaction following the administration of abacavir. Accordingly such patients should not be treated with this nucleoside analogue.

In accordance with a second embodiment of the present invention there is provided a method for detecting a patient's hypersensitivity to abacavir, comprising the step of: screening the patient for at least a marker specific to 57.1AH that is HLA-B*5701 and at least a marker(s) specific for one or more of the Major Histocompatibility Complex markers and HLA subtypes selected from the group consisting of C4A6, HLA-DRB1*0701 (DR7) or HLA-DQB1*0303 (DQ3).

In accordance with a third embodiment of the present invention there is provided a method for detecting a patient's hypersensitivity to abacavir, comprising the steps of: screening the patient for at least a marker specific to 57.1AH that is HLA-B*5701 and one or more markers located between HSP 70.2 and HSP70.1 in 57.1AH, or more preferably 2 kb 3′ of HSP70.1 and 5′ HSP70.2 which are indicators of specificity for abacavir hypersensitivity.

In accordance with a fourth embodiment of the present invention, there is provided a kit for screening a patient(s) for the predisposition to abacavir hypersensitivity.

Other aspects and advantages of the invention will become apparent to those skilled in the art from a review of the ensuing description, which proceeds with reference to the following illustrative drawings.

### BRIEF DESCRIPTION OF DRAWINGS

FIG. 1. Markers of the 57.1 ancestral haplotype are increased in frequency in the abacavir hypersensitive group compared with the control populations, and with the abacavir tolerant group. The presence of the indicated markers are shown by shading. To facilitate comparisons between the groups the vertical axes have been scaled for all groups to show the proportion of patients with the various components of the 57.1 ancestral haplotypes.

FIG. 2. Recombinant mapping of the abacavir hypersensitivity, region in abacavir exposed patients with markers characteristic of the 57.1 ancestral haplotype identifies a putative susceptibility region. For each individual the presence of the marker is indicated by shading. The vertical axis is proportionate to the number of patients. The extent to which the recombinant haplotype extends into the putative region in the abacavir tolerant patients is unknown and therefore has been illustrated with jagged shading.

FIG. 3. Refined recombinant mapping of the abacavir hypersensitivity region in abacavir exposed patients with markers characteristic of the 57.1 ancestral haplotype utilising microsatellite and single nucleotide polymorphism markers between C4A6 and MEGT1 identifies a putative susceptibility region consisting of a 40 kb region extending from Neu1 to Hsp70.1. For each individual the presence of the marker is indicated by shading. The vertical axis is proportionate to the number of patients.

FIG. 4. Refined recombinant mapping of the abacavir hypersensitivity region in abacavir exposed patients with markers characteristic of the 57.1 ancestral haplotype by genomic sequencing of Hsp70.2 and Hsp70.1 genes identifies a putative susceptibility region consisting of a 14 kb region extending from Hsp70.2 to Hsp70.1. For each individual the presence of the marker is indicated by shading. The vertical axis is proportionate to the number of patients.

### DETAILED DISCLOSURE OF THE INVENTION

General

Those skilled in the art will appreciate that the invention described herein is susceptible to variations and modifications other than those specifically described. It is to be understood that the invention includes all such variation and modifications. The invention also includes all of the steps, features, compositions and compounds referred to or indicated in the specification, individually or collectively, and any and all combinations or any two or more of the steps or features.

The present invention is not to be limited in scope by the specific embodiments described herein, which are intended for the purpose of exemplification only. Functionally equivalent products, compositions and methods are clearly within the scope of the invention as described herein.

All references cited, including patents or patent applications are hereby incorporated by reference. No admission is made that any of the references constitute prior art.

Throughout this specification, unless the context requires otherwise, the word “comprise”, or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout.

Description of Preferred Embodiments

The present invention provides methods of analysis, suitable for the identification and determination of a patient's hypersensitivity to abacavir. It provides methods for individualisation of a patient's treatment using such information as well as methods to tailor HIV drug treatment regimes to individual patient needs.

It is known that particular arrangements of alleles, referred to as ancestral haplotypes, are maintained and transmitted through generations en bloc, with a low frequency of recombination events within these genetic blocks. It has been postulated that ancestral haplotypes and their simple recombinants (i.e. a haplotype resulting from historic crossover event(s) between two ancestral haplotypes) account for at least 70% of the observed haplotypes in Caucasian populations.

According to one embodiment, the invention provides a method for identifying whether a patient will exhibit a hypersensitive reaction or like-reaction to treatment with abacavir, comprising the step of: typing the patient for the presence of the 57.1 ancestral haplotype. Individual's that possess the 57.1 ancestral haplotype have been observed to fall into a risk category for abacavir hypersensitivity. Thus, in accordance with the present invention such patient's should not be treated with abacavir.

A patient may be identified as possessing the 57.1 ancestral haplotype by typing the patient for one or more alleles or markers of those alleles that are characteristic of the haplotype.

Those skilled in the field will be aware that the term marker includes any genomic single nucleotide polymorphism, insertions, detections, re-arrangements, variable repeat sequences that encompasses microsatellites, minisatellites or complex repeats, spliced genes and or transcripts or any other change in the genomic sequence or messenger RNA.

Those skilled in the field will be familiar with the alleles that are members of this haplotype and will understand that any one or more of these alleles may be used in the identification process. Some of the common alleles comprising the 57.1 ancestral haplotype include, for example: HLA-DRB1*0701 (DR7), DQB1*0303 (DQ3), D6S1014*137, C4A6, rs419788G (SKI2W), rs437179G (SKI2W), RS494620C (NG22), GenBank Accession number AF134726 nucleotide 34569A (G9A), rs1061581A (Hsp70.2), rs562047G (Hsp70.1), rs1043618G (Hsp70.1), rs2227957C (HspHom), N3_{—}2_{—}5SnRNP*461, N3_{—}2_{—}1 MSH5*421, N3_{—}2 2CLCP*369, D6S273*135, TNF-238A, MICA*194, MIB*344, HLA-B*5701.

Individual alleles making up the 57.1 ancestral haplotype may be either haplospecific (eg HLA-B*5701 and 57.1 AH) or common to multiple ancestral haplotypes (eg HLA-DRB1*0701 represented in 57.1, 47.1 and 13.1 haplotypes). Thus, it is possible that simply detecting the presence of a single allele may not provide sufficient specificity to confirm, with certainty, whether a patient is a member of the 57.1 ancestral haplotype. Accordingly, in a preferred form of the invention the typing will seek to identify at least a single marker that is haplospecific for the 57.1 ancestral haplotype (such as HLA-B*5701) and or a combination of two or more alleles that may be common to multiple ancestral haplotypes but when detected together in one individual are sufficiently distinctive for the 57.1 ancestral haplotype. In a highly preferred form of the invention the patient is screened for markers specific to the 57.1AH, in particular to the HLA subtype HLA-B*5701.

In accordance with a second embodiment of the present invention there is provided a method for detecting a patient's hypersensitivity to abacavir, comprising the step of: screening the patient for at least a marker specific to HLA-B*5701 and at least a marker(s) specific for one or more of the HLA subtypes selected from the group consisting of C4A6, HLA-DRB1*0701 or HLA-DQB1*0303.

Results presented herein establish that a positive association exists between abacavir hypersensitivity and the presence of the 57.1 ancestral haplotype. Thus according to the second embodiment a patient is screened for HLA-B*5701 and one or more HLA subtypes selected from the group consisting of: C4A6, HLA-DRB1*0701 or HLA-DQB1*0303. Such screening provides a surrogate marker for the presence of the entire 57.1 ancestral haplotype and therefore provides a predictive test for abacavir hypersensitivity.

Preferably, the method of the invention is performed by screening a patient to detect the presence of at least HLA subtype HLA-B*5701 and one or more of C4A6, HLA-DRB1*0701 or HLA-DQB1*0303. More preferably, the patient will be HLA typed for HLA-B*5701 and at least C4A6. Alternatively, the patient will be HLA typed for HLA-B*5701 and at least HLA-DRB1*0701. Alternatively, the patient will be HLA typed for HLA-B*5701 and at least HLA-DQB1*0303. In an even more preferred form of the invention the patient will be HLA typed for HLA-B*5701 and at least HLA-DQB1*0303 and HLA-DRB1*0701. In the examples of the invention the patients were subtyped for the presence of: (a) HLA-B*5701, (b) HLA-B*5701, HLA-DRB1*0701 and HLA-DQB1*0303 or (c) HLA-B*5701 and HLA-DQB1*0303.

HLA-B, HLA-DR and HLA-DQ typing is currently clinically available. Those of ordinary skill will be familiar with methods for such typing, which include, for example, microcytotoxicity/serology typing, direct sequence based typing, sequence specific priming (SSP) analysis, sequence specific oligonucleotide (SSO) analysis, reverse sequence specific oligonucleotide (reverse SSO), restriction length fragment polymorphism (RFLP) or amplification length fragment polymorphism (AFLP) typing. The presence of HLA-DRB1*0701 together with HLA-DQB1*0303 indicates the presence of the HLA DR-DQ part of the 57.1 ancestral haplotype. The presence of the HLA-B*5701, HLA-DRB1*0701 and HLA-DQB1*0303 provides a surrogate marker for the presence of the entire 57.1 ancestral haplotype between HLA DR-DQ and HLA-B and therefore provides a predictive test for abacavir hypersensitivity.

In accordance with a third embodiment of the present invention there is provided a method for detecting a patient's hypersensitivity to abacavir, comprising the step of: screening the patient for at least a marker specific to HLA-B*5701 and a selection of markers between the approximate 40 Kb region between Neu1 and HSPHom which markers are specific for abacavir hypersensitivity.

Preferably, the selection of markers is all of the markers between the approximate 40 kb region between Neu 1 and HSP Hom, which markers are specific for abacavir hypersensitivity. More preferably the selection of markers is 80-90% of the markers between the approximate 40 kb region between Neu 1 and HSP Hom, which markers are specific for abacavir hypersensitivity. In a more preferred embodiment the selection of markers is 60-70% of the markers between the approximate 40 kb region between Neu 1 and HSP Hom, which markers are specific for abacavir hypersensitivity. In a highly preferred embodiment, the selection of markers is between 40-50% of the markers between the approximate 40 Kb region between Neu1 and HSP Hom, which markers are specific for abacavir hypersensitivity. In a further preferred embodiment, the selection of markers is 30% of the markers between the approximate 40 Kb region between Neu1 and HSP Hom, which markers are specific for abacavir hypersensitivity. In a highly preferred embodiment, the selection of markers is between 20% of the markers between the approximate 40 Kb region between Neu1 and HSP Hom, which markers are specific for abacavir hypersensitivity. More preferably, the selection of markers is 10% of the markers between the approximate 40 Kb region between Neu1 and HSP Hom, which markers are specific for abacavir hypersensitivity.

In accordance with a further embodiment of the present invention there is provided a method for detecting a patient's hypersensitivity to abacavir, comprising the step of: screening the patient for at least a marker specific to 57.1 AH such as HLA-B*5701 and a selection of markers between the approximate 18 Kb region extending from between 2 kb 3′ of HSP70.2 and 2 Kb 5′ of HSP70.1, with markers that are specific for abacavir hypersensitivity.

Identification of such markers will be achieved by following methods described herein. For example, such markers may be identified by comparing the nucleotide regions in hypersensitive versus tolerant patients in the identified region.

While the present invention is described in the context of detecting specific markers, which present a surrogate marker for the presence of the entire 57.1 ancestral haplotype those skilled in the art will appreciate that, in addition, to the above markers other markers within the 57.1 ancestral haplotype may also be detected, which improve the predictive power of the test for abacavir hypersensitivity. For example, in addition to the above mentioned markers, TNF-238A can be used as a surrogate marker for the 57.1 AH.

In a further embodiment of the invention the predictive power of the test for abacavir hypersensitivity may be improved by also screening for the single nucleotide polymorphism TNF-308A.

In this respect the applicant has observed that TNFα levels appear to increase during hypersensitivity reactions. Also, data so far obtained suggests that the TNF-238A and or TNF-308A alleles, that are believed to be associated with higher TNFα levels, are noticeably more frequent in patients with abacavir hypersensitivity reactions compared to patients who are tolerant of abacavir.

HLA typing may be accomplished by a variety of different methods. Some commonly used methods in the art include, for example, microcytotoxicity/serology, direct sequence based typing, sequence specific priming (SSP), sequence specific oligonucleotide (SSO), reverse sequence specific oligonucleotide (reverse SSO), restriction length fragment polymorphism (RFLP) or amplification length fragment polymorphism (AFLP) typing. HLA subtype determination may also be accomplished by a variety of methods known in the art, which include for example, serological or cellular HLA typing techniques as well as non-serological, DNA based HLA typing methods. Non-serological typing methods are preferentially used in the method of the invention. Such methods include, for example: restriction fragment length polymorphism analysis, sequence specific oligonucleotide probing and/or priming techniques, and DNA sequencing. The polymerase chain reaction (PCR) process may also be used to amplify genomic DNA permitting the usage of more convenient HLA typing procedures. In a highly preferred form of the invention extended Major Histocompatilitiy Complex (MHC) typing is carried out using sequence analysis, including micro-satellite sequence analysis or single nucleotide polymorphism analysis.

In a fourth embodiment of the present invention, there is provided a kit containing components for determining a patient's predisposition to abacavir hypersensitivity. Preferably, the components will include at least a marker specific to HLA-B*5701 and a selection of markers between the approximate 40 Kb region between Neu1 and HspHom which markers are specific for abacavir hypersensitivity.

Where the herein described method(s) is employed to individualise HIV therapy in a patient, the individual is HLA typed for one or more markers or alleles in the 57.1 ancestral haplotype or more preferably for the presence of one or more of the subtypes HLA-B*5701, or (HLA-DRB1*0701 and HLA-DQB1*0303) using sequence based typing. The method may be further enhanced by also performing C4 allotyping for the C4A6 allele by immunofixation electrophoresis. Where a patient is found to display a positive association with either the 57.1 ancestral haplotype or more preferably for the presence of HLA-B*5701 and one or more of the subtypes C4A6, HLA-DRB1*0701 or HLA-DQB1*0303 that patient should be excluded from abacavir therapy and would be treated with another HIV inhibitor.

### BEST MODE(S) FOR CARRYING OUT THE INVENTION

Further features of the present invention are more fully described in the following non-limiting Examples. It is to be understood, however, that this detailed description is included solely for the purposes of exemplifying the present invention. It should not be understood in any way as a restriction on the broad description of the invention as set out above.

### EXAMPLE 1

Subjects: Five hundred and eighty one active participants in the Western Australian HIV Cohort Study on 31 Dec. 2001 were considered. These patients were HLA typed at HLA-A, -B, -C, -DR and -DQ at enrolment into the cohort study and followed-up at one to three monthly intervals. A clinician actively collected details of the anti-retroviral treatment history and adverse drug reactions at each visit. This included a specific question to record any history of possible hypersensitivity to abacavir. The first 200 participants of the cohort prescribed abacavir to 31 Dec. 2001 were included in the study, with abacavir prescription validated in all cohort cases through the use of the Royal Perth Hospital pharmacy database. The medical records of abacavir-exposed individuals were reviewed by a single clinician blinded to HLA typing results for evidence of abacavir hypersensitivity, utilising standardised diagnostic criteria:
Onset of at least two of the following symptoms within 6 weeks of abacavir initiation: fever, rash, gastrointestinal symptoms (nausea, vomiting, diarrhoea or abdominal pain), lethargy, malaise, arthralgia, myalgia or respiratory symptoms (dyspnoea, sore throat or cough)
Resolution of symptoms within 72 hours of discontinuation of abacavir, and
Absence of an alternative likely explanation of the symptoms.

Cases were reviewed and classified as either: (1) definite cases of abacavir hypersensitivity; (2) cases in which abacavir hypersensitivity could be excluded, where abacavir therapy had been continuous for ≧6 weeks (abacavir tolerant); or (3) cases in which abacavir hypersensitivity could not be excluded because symptoms experienced in the first 6 weeks of abacavir exposure did not meet diagnostic criteria (abacavir hypersensitivity not excluded). Cases from group 3 were excluded from analyses of HLA allele frequency and abacavir hypersensitivity, but were included in calculations of positive and negative predictive values of the presence of HLA alleles for abacavir hypersensitivity.

Additional non-HIV infected control subjects were recruited from the Western Australian bone marrow donor registry (n=3212). As a requirement of the registry, all individuals were typed at HLA-A and -B by standard microcytotoxicity assays and at HLA-DR loci by either serological or sequence based typing methods. Allele frequencies were also examined in 381 HIV-infected, non-abacavir-treated controls.

HLA Typing

HLA-A, -B, and -C typing was performed by standard microcytotoxicity assays. HLA-B and HLA-C sequencing analysis was also performed in cases where serological methods were insufficient to resolve specific alleles (for example, HLA-B17 was resolved to HLA-B*5701 and -B*5801 in all cases). HLA-DRB1 and -DQ typing was performed by sequence analysis.

Assignment of Components of the 57.1 AH

This study exploits current knowledge of the MHC and the alleles of polymorphic microsatellite and single nucleotide polymorphism (SNP) markers. Initial data obtained in the study indicated an association between abacavir hypersensitivity and HLA alleles specific for the 57.1AH. In view of this the inventors sought to define the boundaries of the abacavir hypersensitivity susceptibility region through the study of subjects with recombinant 57.1 AHs. The 57.1AH includes HLA-B*5701, C4A6, HLA-DRB1*0701(DR7), and -DQB1*0303(DQ3). HLA-DR-DQ haplotypes show extremely high linkage disequilibrium in all populations but DR7 may be associated with either DQ3 (on the 57.1 ancestral haplotype) or DQ2 (on other haplotypes). Therefore, the combination of HLA-DR7 plus -DQ3 in individuals who did not have HLA-DQ2 was necessary for the assignment of a recombinant 57.1AH.

SNP and Microsatellite Typing

Samples were typed for the TNFA-238A/G and 308A/G substitutions, as previously described in Wilson A G, et al., (1992) “Single base change in the human tumour necrosis factor alpha (TNFA) gene detectable by NcoI restriction of PCR product.” Hum Mol Genet, 1:353. Four microsatellites spanning the central MHC region (D6S1014, D6S273, MIB, and MICA) were used for mapping this region, as previously described in Cheong K Y, et al (2001) “Localization of central MHC genes influencing type I diabetes.” Hum Immunol, 62:1363-1370.

Statistical Analyses

P-values were corrected for comparisons of multiple HLA alleles (P_{c}) by dividing the raw P-value by 35 (ie estimated number of HLA alleles present within the most polymorphic locus (HLA-B)). Odds ratios (OR) were calculated using Woolfs method with Haldane's modification [Svejgaard A, and Ryder L P (1994) “HLA and disease associations: detecting the strongest association.” Tissue Antigens, 43:18-27]. Comparisons of demographic and related data in abacavir hypersensitivity (n=18) and abacavir tolerant (n=167) groups were performed using Fisher's Exact tests for dichotomous variables, and t-tests for continuous variables.

Prevalence of Abacavir Hypersensitivity

In the cohort of 200 individuals, 18 definite cases of abacavir hypersensitivity were identified, while 167 individuals had more than six weeks exposure to abacavir without developing hypersensitivity (abacavir tolerant), and 15 individuals experienced non-specific symptoms during the first six weeks of abacavir exposure but did not meet the criteria for definite abacavir hypersensitivity. Hence, the prevalence of abacavir hypersensitivity in our population was 9%.

Patient demographics, antiretroviral therapy exposure, and immunological status (CD4 T cell proportion and absolute number) in the three study groups are presented in Table 1. The proportion of Caucasoids in the abacavir hypersensitive group was significantly higher than in the tolerant group (100% versus 82%, P=0.03), while the groups were similar in terms of other variables considered. Within the ABC-exposed cohort (n=200), 176 participants were classified as Caucasoid, while the non-Caucasoid group consisted of 7 Africans, 11 indigenous Australians, and 6 Asians.

TABLE 1
Demographic variables, antiretroviral exposure, and immunological
status in abacavir hypersensitive and abacavir tolerant groups.

| | Abacavir hypersensitive | Abacavir tolerant | |
|---|---|---|---|
| | (n = 18) | (n = 167) | P-value |
| SEX | | | |
| Males | 88.9% | 86.8% | 1.0 |
| Females | 11.1% | 13.2% | |
| RACE | | | |
| Caucasoid | 100.0% | 88.0% | 0.23 |
| Non-Caucasoid | 0.0% | 12.0% | |
| AGE (years) | | | |
| Mean | 44.8 | 42.8 | 0.43 |
| Std Err | 2.1 | 0.8 | |
| CD4% | | | |
| Mean | 19.8 | 20.7 | 0.71 |
| Std Err | 1.9 | 0.8 | |
| absolute CD4 | | | |
| Mean | 444 | 442 | 0.98 |
| Std Err | 55 | 23 | |
| ART naïve | | | |
| naïve | 11.1% | 15.6% | 1.0 |

HLA Allele Frequency

There were several striking differences in the frequency of HLA alleles in the abacavir hypersensitivity group compared to the abacavir tolerant group, presented in Table 2.

TABLE 2
Contribution of combined or individual loci of 57.1 ancestral
haplotype to abacavir hypersensitivity susceptibility

| | Abacavir | Abacavir | | |
|---|---|---|---|---|
| | hypersensitive | tolerant | | Odds |
| Locus | (n = 18) | (n = 167) | P_{c} value | Ratio |
| HLA-B*5701, -DR7, -DQ3 | 13 (72%) | 0 (0%) | <0.0001 | 822 |
| HLA-B*5701 | 14 (78%) | 4 (2.4%) | <0.0001 | 117 |
| HLA-DR7, -DQ3 | 13 (72%) | 5 (3%) | <0.0001 | 72 |

HLA-B*5701 was present in 78% of cases of abacavir hypersensitivity and 2.3% abacavir tolerant (OR 117, P_{c}<0.0001), while the combination of HLA-DRB1*0701 and -DQ3 was present in 72% cases of abacavir hypersensitivity and 3.0% abacavir tolerant (OR=72, P_{c}<0.0001). The HLA-B*5701, HLA-DRB1*0701, -DQ3 haplotype, present in 72% of abacavir hypersensitivity cases and 0% of the abacavir tolerant (OR=822, P_{c}<0.0001), was found to be more predictive of the clinical syndrome than its component alleles. Within the entire abacavir-exposed cohort (n=200), the presence of HLA-B*5701 and HLA-DRB1*0701, -DQ3 alleles had a positive predictive value for abacavir hypersensitivity of 100%, and a negative predictive value of 97.3%.

In this cohort, all cases of abacavir hypersensitivity were observed in Caucasoids. The presence of HLA-B*5701 and/or HLA-DRB1*0701 alleles was also only found among Caucasoids. Repeating the analyses in a dataset restricted to Caucasoid participants (n=176) revealed an increased frequency of HLA-B*5701, (OR=103, P_{c}<0.0001), -DRB1*0701 plus -DQ3 (OR=64, P_{c}<0.0001), and the combined alleles (OR=724, P_{c}<0.0001), among cases of abacavir hypersensitivity compared with the abacavir tolerant group. The presence of the HLA-B*5701, HLA-DRB1*0701, -DQ3 haplotype was associated with positive and negative predictive values of 100% and 96.9%, respectively.

Frequency of Complete or Partial 57.1 Ancestral Haplotype in Control Populations

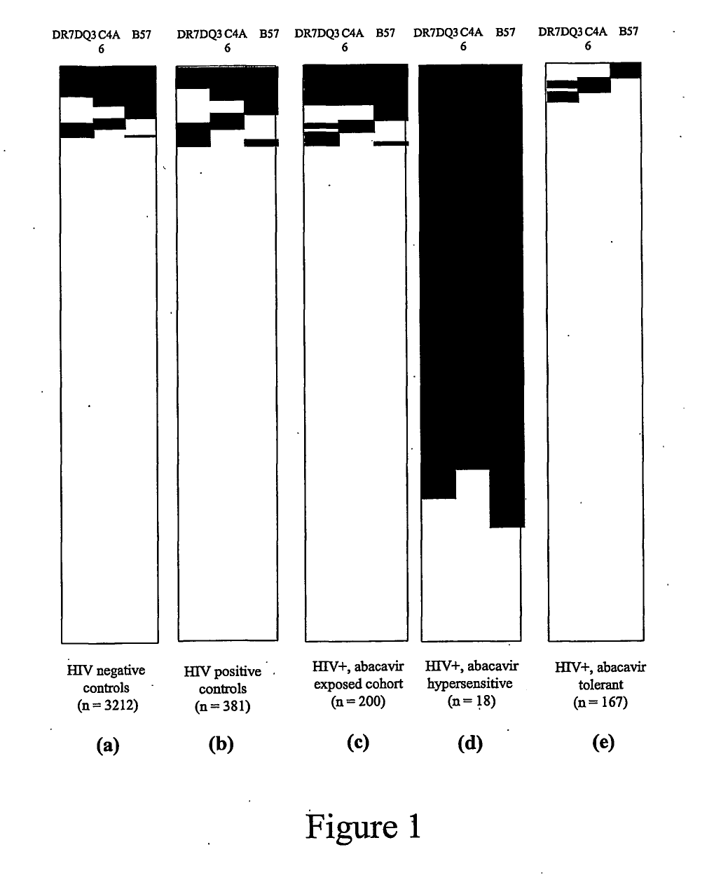

The frequency of the C4A6 allele, an additional specific marker of the 57.1 ancestral haplotype within the central MHC, was assessed in the abacavir-exposed groups, as well as in two control populations (381 HIV infected patients not exposed to abacavir and 3212 HIV negative bone marrow donors). The distribution of the HLA-B*5701, C4A6, and HLA-DRB1*0701 plus -DQ3 alleles of the 57.1 ancestral haplotype was similar in the three groups (FIGS. 1a,b,c). HLA-B*5701 was found in 9.0% of HIV-negative controls and 8.4% of HIV-infected controls, compared with 9.5% in the abacavir-exposed cohort. The combination of 57.1 haplospecific markers HLA-B*5701, -C4A6, -DR7, and -DQ3 was found in 4.2% and 3.2% of these two control populations, respectively, compared with 7% in the abacavir-exposed cohort. In contrast, alleles of the 57.1 ancestral haplotype were greatly over represented in the abacavir hypersensitive as compared to the tolerant group, as represented in FIGS. 1d and 1e.

Mapping of Putative Susceptibility Loci Within the MHC

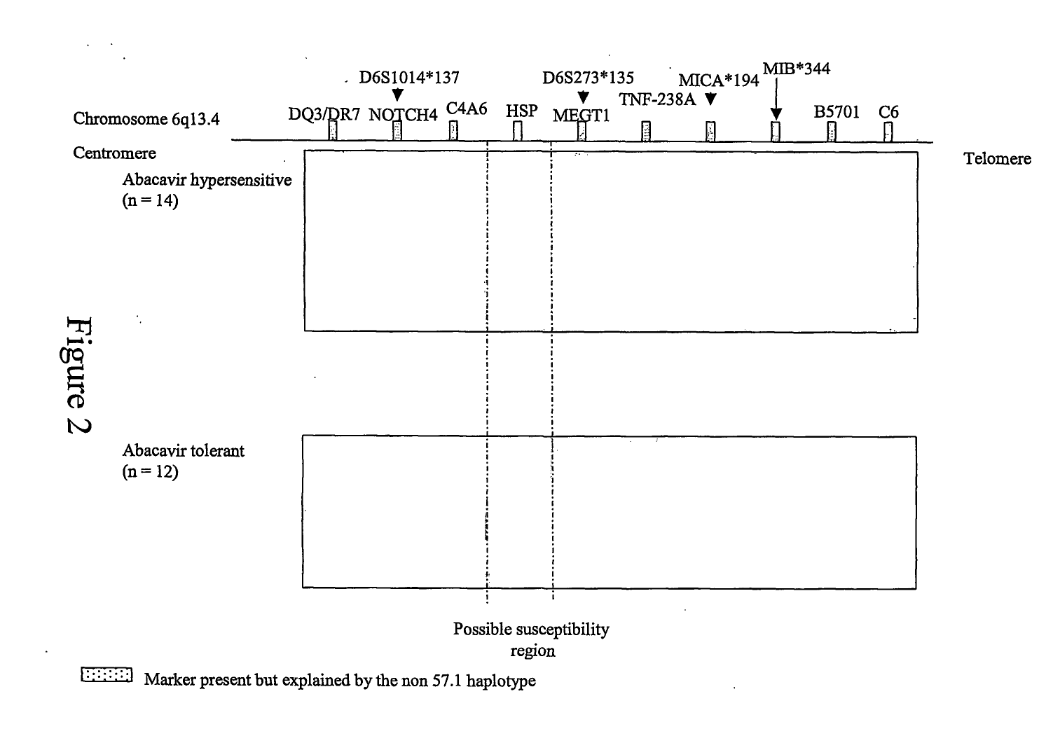

Central MHC markers characteristic of the 57.1 ancestral haplotype (D6S1014*137, C4A6, D6S273*135, TNF-238A, MICA*194, MIB*344) were examined to confirm the presence of the haplotype and to map the extent of the recombinant haplotypes in cases and controls (FIG. 2). Mapping of recombinant 57.1 haplotypes among abacavir hypersensitive and abacavir tolerant cases identified a ˜300 kB candidate region telomeric to the C4A6 allele that was unique to abacavir hypersensitivity cases in the cohort, thus identifying the most parsimonious susceptibility region. The telomeric boundary of this region was marked by the D6S273*135 microsatellite present in intron 1 of Megakaryocyte-enhanced gene transcript-1 (MEGT1/G6d). Among the 14 abacavir hypersensitivity cases with the HLA-B*5701, -DRB1*0701, -DQ3 haplotype, carriage of all markers between C4A6 and HLA-Cw6 was found (ie D6S273*135, TNF-238A, MICA*194, MIB*344, HLA-B*5701), consistent with the presence of non-recombinant 57.1 ancestral haplotype in this region. Hence, presence of 57.1 ancestral haplotype alleles in a region identified by the presence of C4A6 at the centromeric boundary, and telomeric of HLA-Cw6, provided sufficient conditions for abacavir hypersensitivity susceptibility in this group (FIG. 2).

Observations

The major finding in this study of 200 consecutive abacavir-treated individuals in the Western Australian HIV Cohort is that the presence of the HLA-B*5701, -DRB1*0701, -DQ3 haplotype is strongly associated with susceptibility to abacavir hypersensitivity, a serious and potentially life-threatening clinical syndrome encountered in approximately 5% of abacavir-treated patients. The presence of this allelic combination is associated with markedly increased risk of developing the syndrome, with an odds ratio comparable to that observed in the association between HLA-B27 and ankylosing spondylitis.

Current practice guidelines for the prevention and management of abacavir hypersensitivity focus on early recognition of suggestive symptoms in the appropriate interval after initiation of abacavir therapy, and prompt cessation of abacavir following diagnosis. The positive and negative predictive values (100% and 97.3%, respectively) associated with the presence of the HLA-B*5701, -DRB1*0701, -DQ3 haplotype in this study support an important and immediately applicable clinical role for HLA typing in this setting.

The findings presented herein are consistent with a direct role for MHC-specific alleles in the pathogenesis of abacavir hypersensitivity. It is possible that the susceptibility locus or loci marked by the presence of HLA-B*5701, -DRB1*0701, -DQ3 participate directly in abacavir-specific antigen recognition by the immune system (ie αβ and/or γδ T cells). This possibility is supported by the existence of a complex intracellular pathway for the metabolism of abacavir, in which this carbocyclic guanine derivative is converted to carbovir monophosphate via multiple pathways involving deamination and phosphorylation steps, prior to undergoing kinase-mediated activation to the active carbovir triphosphate form. The numerous metabolites thus formed may then undergo haptenation to endogenous proteins to achieve antigenicity, or may bind directly to MHC molecules to elicit T cell activation, consistent with the proposed pathogenesis of a number of idiosyncratic drug reactions. In relation to recombinant mapping of candidate susceptibility regions, the most parsimonious region identified contains several central MHC genes, including a family of heat shock protein genes that may be involved in antigen chaperoning and folding as well as providing direct immunostimulatory signals in the presence of antigen. However, it is notable that a larger region defined by C4A6 and HLA-Cw6 on the 57.1 haplotype provides sufficient conditions for abacavir hypersensitivity susceptibility, raising the possibility that two or more loci on this region of the 57.1AH (including HLA-B*5701 itself, as a potential mediator of Class I-restricted antigen presentation) may cooperate to induce susceptibility.

Mapping of individual susceptibility genes that are marked by the presence of HLA-B*5701, -DRB1*0701, and -DQ3 requires the identification of informative abacavir hypersensitive and abacavir tolerant cases with recombinant 57.1 haplotypes, as the extremely high linkage disequilibrium of MHC alleles within the 57.1 haplotype may otherwise hamper localisation of involved susceptibility loci. In this way, markers that are haplospecific but distant from a true susceptibility locus may display stronger linkage disequilibrium than non-haplospecific markers that are more proximate to it, thereby confounding multivariate statistical analyses that assume linkage disequilibrium to be primarily attributable to genetic distance.

The strong association between abacavir hypersensitivity and HLA-B*5701, -DRB1*0701, and -DQ3 identified in this study provides a basis for the development of a test for abacavir hypersensitivity, as well as for an increased understanding of the pathogenesis of this potentially life-threatening clinical syndrome.

### EXAMPLE 2

### Refined Mapping of Genetic Susceptibility to Abacavir Hypersensitivity

Summary: As described above, a region of approximately 300 kb between C4A6 and D6S273*135 (MEGT1) was identified as a region likely to carry the gene(s) contributing to abacavir hypersensitivity. In addition, a number of other idiosyncratic drug reactions have been shown to involve MHC-restricted presentation of the drug or its reactive metabolite after peptide haptenation or direct covalent or non-covalent binding of the drug to HLA molecules. Therefore, the inventors examined the association of MHC alleles within the 300 kb interval bound by C4A6 and the D6S273*135 microsatellite allele in a restricted patient set recombinant for the 57.1 AH. Gene(s) likely to initiate the hypersensitive response independently or through HLA-B5701 restricted presentation to the immune system were examined.

Further mapping as described below narrowed the susceptibility region to a 40 kb region bounded by Neu1 and 70.1 and then further to a 14 kb region bounded by HSP70.2/rs539689C and HSP70.1/-27G. It appears that any haplospecific combination of alleles for the 57.1 AH in this latter region will be a preferred form of the test for ABC HSR.

Subjects: A restricted patient set from the 249 abacavir exposed participants from the Western Australian HIV cohort study were examined. The patients included HIV+, abacavir hypersensitive (ABC HSR) patients (n=20) and HIV+, abacavir tolerant (ABC non-HSR) individuals (n=12). These patients carried the entire or partial 57.1 AH in the MHC region.

Method: Polymorphic satellites markers in G6e, MSH5, CLCP, SnRNP, NEU1, and C2 were typed using fragment length polymorphism analysis. Briefly, the amplified products were diluted 1 in 10 to 1 in 25 and mixed with formamide (10 μL) and GS 1000 ROX (0.5 μL). The products were injected at 15 mV and electrophoresed at 15 mV for 30 minutes using ABI 3100 gene scan application (Applied Biosystems, Foster City, Calif.). Alleles were assigned on the basis of size by comparison with the molecular weight standards.

46 SNPs were examined within the 300 kb, C4A6 to D6S273 interval using RFLP, sequence—(ABI 3100) or pyro-sequencing-based (Pyrosequencer, Millenium Science) typing methods. This data was used to generate haplotype maps in the sample set to identify the boundaries of the susceptibility region and the candidate genes contributing to susceptibility to abacavir hypersensitivity. The SNPs typed by the RFLP method included the rs2227957CT (Hsp70Hom) where the T allele was NcoI restricted and rs1061581GA (Hsp70.2) where the A allele was PstI restricted. The other SNPs identified by sequencing include those in SKI2W: rs519788GA, rs437179GT; RD: rs2072632CT; DOM3L: rs2743395AT; Bf: rs1048709CT, rs537160CT; C2: rs22452572AG, rs1042664CG; NG36: GenBank Accession No. AF134726: nt21232AG; BAT8: rs7887CA, rs21265CT, rs589428GT, GenBank Accession No. AF134726:nt21256CA, nt21345GC, nt21346CT, nt37509AG; NG22: rs570262GA, rs494620CT, IMS-JST014989CT, GenBank Accession No. AF134726: nt34726AG; G9 rs1802189AG, GenBank Accession No. AF134726AG, GenBank Accession No. AF134726:nt91310AT; Hsp70.2: rs1043619GC, rs1043620CT, rs562047GC, rs506770GC, rs541340TG, rs539689G; Hsp70.1: rs1043619GC, rs1043620CT, rs562047GC, −27GC, +116CT, +242GA, +351GA, +505CT, +565CG, Hsp70hom: +404TG, +423AC, +424CA, +462TC, rs2227957CT, rs2075800GA. (The + number refers to the amino acid residue in the mature Hsp70 protein (http://www.ncbi.nlm.nih.gov/SNP/snp_ref:cgi?locusld=3304;3303).

Initially, direct sequencing of the 57.1 AH between HSP70.2 and HspHom was undertaken to identify all the SNPs present on the 57.1 AH to be used as a genetic test to predict the development of HSR. Further direct sequencing of Hsp70.1 and Hsp70.2 genes was done using nested PCRs with primers that amplified the full length gene followed with six overlapping amplifications.

More specifically, pyrosequencing primers were designed to genotype all the identified SNPs carried out on the 57.1 AH, including the SNPs in HSP70.1 and HSP70.2 region. SNP typing of the entire 249 patients was carried out by sequencing using the available primers used in the sequencing of the 57.1 AH. Table 3 lists the primers and conditions used to sequence the 14 kb HSP70.2 to HSP70.1 region of the 57.1 AH.

Briefly, the conditions for the PCR reaction included 100 ng DNA, 1 mM MgCl2 Tris. HCl pH8.3, 4 mM dNTPs, 0,2 μl ampliTaq, The amplifications include 1 cycle at 95° C. for 5 min, 35 cycles at 95° C. for 30 sec, 60° C. for 30 sec, 72° C. for 1 min followed by 1 cycle at 4° C.

TABLE 3
Primers used for genomic sequencing of the 57.1 AH

| | | | | |
|---|---|---|---|---|
| | | NP-1st | Annealing | MgCl₂ |
| F primer | R primer | round primers | temp | conc mM |

| | | | | | | |
|---|---|---|---|---|---|---|
| 77116 | CAACATGGATGAAACCTG | 75880 | CTCTGAGGCCTATGGAGA | | 60 | 2.5 |
| 77868 | CATCTTCAGCCTGACTG | 76611 | CACAGCGGTGTTCAACC | | 60 | 1.5 |
| 79123 | TGCGCTTCAGCGCCTAA | 77806 | AGACATCAGCCTCCACA | | 53 | 2.5 |
| 79989 | catggacgagatctcctc | 77806 | AGACATCAGCCTCCACA | | | |
| 79989 | catggacgagatctcctc | 78983 | gtatctccattgtaacgt | | | |
| 82317 | aggctaaacccaatcagcat | 81448 | gaaggagctggagcaggtgt | 83990/81448 | 55 | 1.5, 2.5 |
| 82815 | CTTCTAGGATGCTAGCG | 81448 | gaaggagctggagcaggtgt | | | |
| 82815 | CTTCTAGGATGCTAGCG | 81878 | AGGGTCTGCATAGGTTG | | 50 | 1.5 |
| 83990 | tgctactgcagctctga | 82747 | AAGTACGCCAGTGAGCA | 82815/86384 | | |
| 83990 | tgctactgcagctctga | 83074 | tggtgtgcgcctgtaat | 82815/86384 | 60 | 2.5 |
| 84894 | gaccaggaataacagtaag | 83929 | ctgtagtactgtggactct | 82815/86384 | 60 | 2.5 |
| 85775 | tcagatgcattgggtgg | 84678 | gagaatgcagtgccacc | 82815/86384 | 60 | 2.5 |
| 86638 | gaagttcagtggcttga | 85593 | aaccgagaatcactcct | 82815/85593 | 60 | 2.5 |
| 87389 | tctagaggcaggcattct | 86384 | ccatggtcttgagtccg | 87389/85593 | 55 | 2.5 |
| 88687 | TCACTGTCTCAACTGCCTAG | 87358 | aaatggccagggttctcttt | 87389/85593 | 60 | 2.5 |
| 89145 | tactggcaggtctggat | 88004 | actctgagctctgatcc | 89145/86384 | 50 | 1.5 |
| 89828 | atcctagattccaacttcct | 88591 | ATAGCACTGTAGACTCTGAC | 89828/88004 | 55 | 1.5 |
| 90835 | tcagctgcaaccaggag | 89635 | cgccatggcaatgaggc | | 55 | 1.5 |
| 91536 | caccaagctcgatgaggctg | 90614 | cagggtcaccatcttgttaa | | 55 | 2 |
| 92139 | TGTAGCTCACCTGCACCTTG | 91141 | TCTCAGGCAGACTAGGCCAT | 92844/91141 | 55 | 1.5 |
| 92844 | AGGACCAGGTCGTGAATCTG | 91770 | TCCGAAGGACTGAGCTCTTG | | 55 | 1.5 |
| 93807 | GCAGCAAAGTCCTTGAGTCC | 91141 | TCTCAGGCAGACTAGGCCAT | 94180/91770 | 55 | 2.5 |
| 94180 | TTCAGACACTATCCCTCCGC | 93083 | GATGACTGCCCTGATCAAGC | 94180/91141 | 55 | 2.5 |

The method involved specific amplification of the HSP70.2 and HSP70.1 genes, the inter-genic region and the 5′ to 3′ regions of the two genes. A nested PCR amplification with specific primers to amplify the first round DNA followed by a second round amplification using pyrosequencing was performed. Table 4 lists the primers used for the genetic typing.

Pyrosequencing Assay

The conditions used for the pyrosequencing assay are as follows. Initial amplification conditions were similar to that described above. The SNP detection by the pyrosequencer PSQ HS 96 (Millenium Science, Sweden) included mixing of the amplified product (10 μl) with sepharose beads (2 μl) in binding buffer (40 μl). Adsorption of the DNA to the beads occurred by shaking for 5 minutes under the following settings: QuantiFERON™, CSL Biosciences, amplitude setting: 7.

The PCR product absorbed to the beads was aspirated using a pyro-sequencer vacuum tool attached to a Millipore vacuum pump, followed by a series of washes in 70% ethanol, denaturing buffer (Tris 1.21 g/L, NaCl 117 g/L, EDTA 0.0292 g/L, Tween 1 ml, pH 7.6), and wash buffer (10 mM Tris acetate pH7.6). The beads were released into a mixture of pyrosequencing primer (0.033 mM) in annealing buffer (Tris 2.42 g/L, 0.43 g/L magnesium acetate tetra hydrate, pH7.6, adjusted with 4M acetic acid). The PCR product was denaturation by heating the plate at 94° C. for 2 minutes. SNPs were identified by using pyrosequencing software.

Results: Initially to identify the primary gene(s) contributing to abacavir hypersensitivity on the 57.1 AH, a series of 46 SNPs and microsatellite markers in the C4A6 to MEGT1 interval were studied. Genetic typing of microsatellites markers characteristic of the 57.1AH was determined using MHC homozygous cell lines (Table 5).

The putative susceptibility region conferring hypersensitivity to abacavir by recombinant mapping and haplotype architecture was refined using polymorphic

TABLE 4
Primers used for SNP detection on the Pyrosequencer

| | | | |
|---|---|---|---|
| Name | F primer | R primer | Pyrosequencing primer |

| | | | |
|---|---|---|---|
| rs1043618 | Biotin-5′-TGTAAAACGACGGCCAGTAGAGCGAAC | 5′-AGGTCGATGCCGATCGCC | 5′-GCTCTCTGTCGGCTC |
| | CTGTGCGGCT | | |
| rs1043619_20 | 5′-TGTAAAACGACGGCCAGTcgcagaacaccgtgtt | Biotin-5′-GTCACGAAGTACAGGCTGT | 5′-TTGACGCGAAGCG |
| rs562047 | 5′-TGTAAAACGACGGCCAGTTGATCACCGCGTTGGT | Biotin-5′-GTGCAGTCGGACATGAAGCA | 5′-GGGGTAGAATGCCTTG |
| | CACC | | |
| rs1061581 | Biotin-5′-TGTAAAACGACGGCCAGTAGGCTCTGC | 5′-GTAGGCCACAGCCTCGTCG | 5′-GCCCGTTGAAGAAGTC |
| | GCGACGCCA | | |
| rs483638 | Biotin-5′-TGTAAAACGACGGCCAGTCAGATCGAG | 5′-GCTGACACCCTCTCGCGCT | 5′-GTTGGTGATGGTGATCT |
| | GTGACCTTCG | | |
| rs506770 | 5′-TGTAAAACGACGGCCAGTCATGAAGAGCGCCGTGG | Biotin-5′-CTCGTCCTTCTCGGGCCAAG | 5′-CAAGATCAGCGAGGC |
| | A | | |
| rs2607020 | 5′-GACGGGATCCGCGACAAG | Biotin-5′-TCGTCTTTCGAGAGTGACTC | 5′-CGCCGGACACTCAAC |
| rs539689 | Biotin-5′-GGTAGTAGTCGCCTGACATG | 5′-GATTAGGTGGAGGAGTTA | |
| HSP70_505 | Biotin-5′-CACGGCCACGGACAAGAG | 5′-TCTCGCGCTGCACCTCGTC | 5′-GGCGGCCCTTGTC |
| Hsp70.1_242 | 5′-GAGGACTTTGACAACAGGC | Biotin-5′-GAGTCGATCTCCAGGCTGG | 5′-GGCTGGTGAACCACTT |
| HSP70_565 | 5′-TCAGCCAAGAACGCCCTG | Biotin-5′-CTCGTCCTTCTCGGCCA | 5′-AACGCCCTGGAGTC |

microsatellite and SNPs. Construction of a 57.1AH map was observed in 15 HLA-B*5701 positive HSR patients. The entire 57.1 AH was observed in 15 HLA-B*5701 positive HSR patients. The presence of non 57.1 AH alleles, NG22/rs494620T, HSP70.2/rs539689G, HSP70.1/-27C, HSP70.1/+242A, HSP Hom/rs2227956T was evidence of the decay of the 57.1 AH in the recombinant patients and tolerant controls.

Using this data the inventors narrowed the susceptibility region to a 14 kb region extending from HSP70.2 to HSP70.1 genes associated with HSR. Direct sequencing of these genes in the 57.1 AH homozygous cell line (DBB) and comparison with the already sequenced 7.1, 8.1 and 18.1 haplotypes has identified a 14 kb haplotype comprising of at least 26 SNPs. The combination of the SNPs on the 57.1 AH between HSP70.2/rs539689G and HSP70.1/-27C will serve as a predictive test for abacavir hypersensitivity.

The SNP alleles of the 57.1AH were determined by sequence of the homozygous cell line DBB. Polymorphisms characteristics at the entire 57.1AH between C4A6 TO D6S273 were observed in the 12 HLA-B*5701+, DR7, DQ3+HSR individuals (HSR patients #1 to #12) as well as in the HLA-B*5701+C4A6+HSR patient #14 and in the HLA-B*5701+, DR7,DQ3+, C4A6-HSR patient #13.

The rs2227957TT non-57.1AH allele seen in HSR patient #13 marked the telomeric end of the putative susceptibility region while the HLA-B*5701+, C4A6-HSR patient #15 carried a non-57.1AH rs494620TT allele in NG22 thereby marking the centromeric end of the susceptibility region in the HSR patients.

Decay of the 57.1AH, from the telomeric end of the interval in the recombinant tolerant nonHSR patients, was observed using a combination of the microsatallite alleles of the 57.1AH (G6E*300, MSH5*421, CLCP*369, SnRNP*462). The HLA-B*5701+nonHSR patient #23 carried the non 57.1 AH alleles in SK12W: rs419788M and rs437179TT and in Hsp70.2 rs539689CC and hence lacked the 57.1 AH block extending from SK12W and Hsp70.2. In addition, the HLA-B*5701−, DR7DQ3+, C4A6+nonHSR patient #27 carried a non 57.1AH block extending from G9A to Hsp70.1.

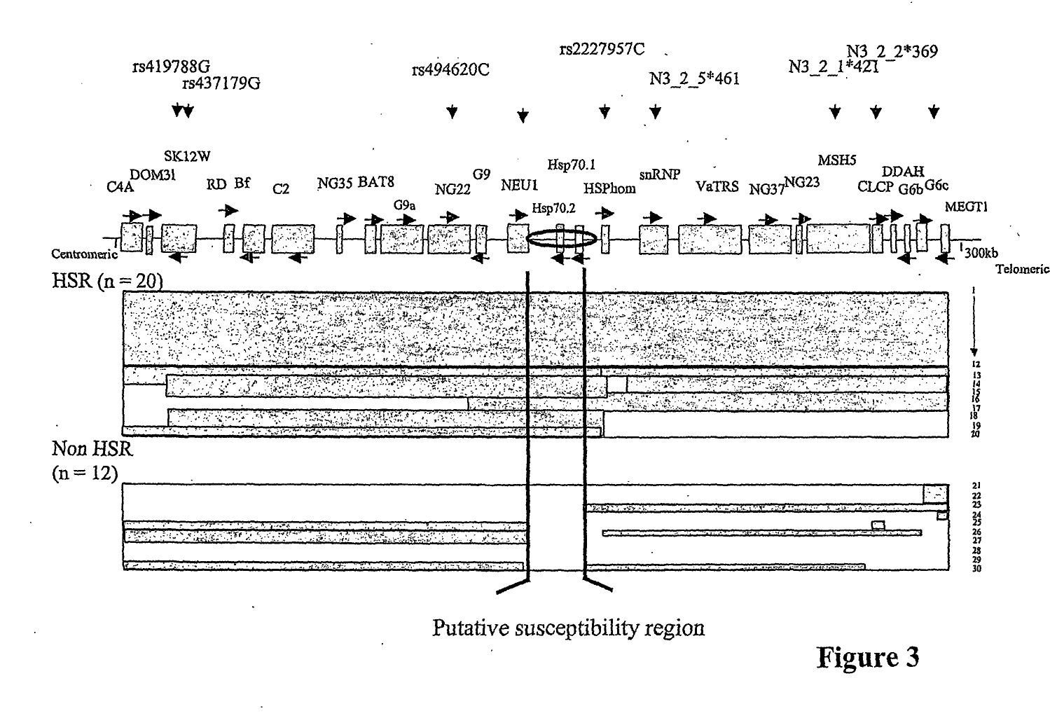

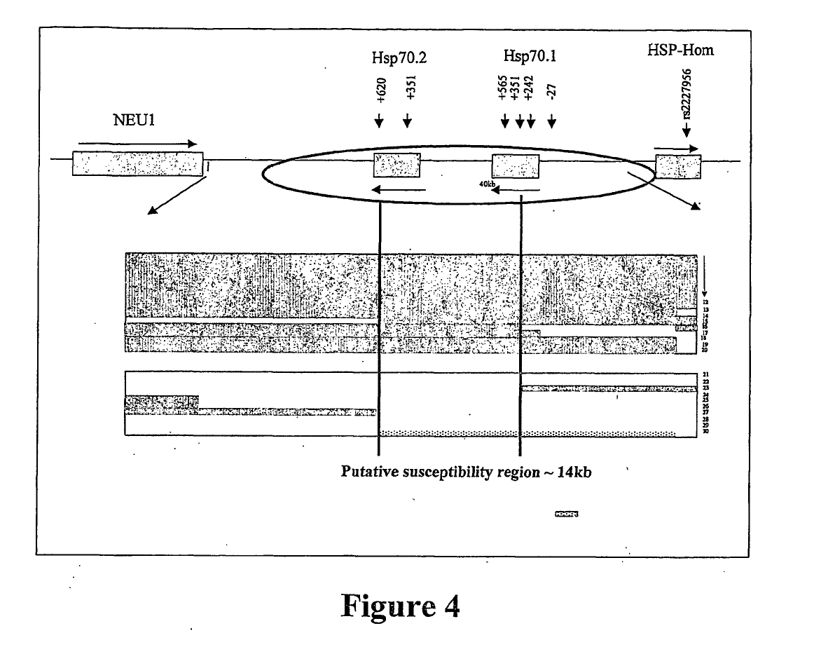

Construction of a map of conserved alleles specific to the 57.1 AH defined the 57.1 AH and determined the decay of this haplotype in recombinant patients and controls (FIG. 3). This has enabled us to refine the map to the smallest region consisting of a 14 kb region extending from HSP70.2 to Hsp70.1. (FIG. 4).

Sequencing of the HSP70 genes present on the 57.1AH homozygous cell line and a typical HSR patient (# 2) and 2 tolerant controls (#23 and #27) has enabled the identification of a 57.1 AH specific SNP in HSP70.2. Table 6 lists the nucleotide variation seen in the subjects examined. The Hsp70.1 sequence was identical in the non HSR patient carrying the HLA-B*5701 compared to the DBB cell thus eliminating the gene as a likely candidate.

TABLE 5
Microsatellites and SNP alleles present on ancestral haplotypes

| | | | | | | SK12W | 70.2 | 70.1 | | |
|---|---|---|---|---|---|---|---|---|---|---|
| | | HLA | HLA | D6S | | rs4371 | rs106158 | rs104361 | 70.1 | Hom |
| AH | n | DQB1 | DRB1 | 1014 | C4A | 79 | 1 | 8 | rs562047 | rs2227956 |
| 7.1 | 4 | 0602 | 1501 | 140 | 3 | G | A | G | G | C |
| 7.2 | 2 | 0501 | 0101 | 125 | 3 + 3 | G | A | G | G | T |
| 8.1 | 4 | 0201 | 0301 | 146 | Q0 | * | G | C | G | T |
| 13.1 | 1 | 0201 | 0700 | 149 | 3 | * | G | G | G | T |
| 18.2 | 4 | 0201 | 0301 | 140 | 3 | G | G | C | C | T |
| 38.1 | 1 | 0302 | 0402 | 125 | 2 | * | * | * | G | T |
| 42.1 | 1 | 0402 | 0302 | 137 | 12 + 91 | G | * | * | * | T |
| 46.2 | 1 | 0601 | 08032 | 143 | 4 | * | A | * | * | T |
| 47.1 | 1 | 0201 | 0700 | 146 | 1 | * | A | * | G | T |
| ? | 1 | 7 | 16 | 140 | 3 | G | G | * | * | T |
| ? | 1 | 6 | 15 | 146 | | T | G | C | C | T |
| 57.1 | 1 | 03032 | 0700 | 137 | 6 | G | A | G | G | C |
| 60.1 | 1 | 0302 | 0404 | 140 | 3 | * | A | G | G | T |
| 62.1 | 2 | 0302 | 0401 | 140 | 3 | * | A | G | G | T |

| N3_2 | | | | | | | | |
|---|---|---|---|---|---|---|---|---|
| _5 | N3_2 | N3_2 | | | | | | |
| SnRN | _1 | _2 | D6S | | | HLA- | HLA- | |
| P | MSH5 | CLCP | 273 | MIB | HLA-B | C | A | ID (10IHW/4AOH |
| 459 | 417 | 375 | 135 | 336 | 7 | 7 | 3 | 9013, 9017, 9081, 9082 |
| 469 | 415 | 371 | 133 | 336 | 7 | 7 | 24 | 9001, 100043X |
| 467 | 407 | 371 | 139 | 350 | 8 | 7 | 1 | 9022, 9086, 9087, 100044V |
| 459 | 423 | 371 | 129 | 336 | 13 | 6 | 30 | 9096 |
| 469 | 419 | 375 | 129 | 326 | 18 | 5 | 30 | 9018, 9019, 9020, 9085 |
| 465 | 416 | 373 | 133 | 332 | 38 | — | 26 | 9026 |
| 467 | 423 | 375 | 127 | 326 | 42 | 2 | 30 | 9021 |
| 461 | 417 | 377 | 135 | 326 | 46 | 11 | 2 | 9066 |
| 461 | 417 | 375 | 131 | 354 | 47 | 6 | 3 | 9047 |
| 459 | * | 369 | * | * | 51 | — | 2 | 9016 |
| 461 | 427 | 363 | 131 | 348 | 53 | 4 | 28 | 9010 |
| 461/3 | 421 | 369 | 135 | 344 | 5701 | 6 | 2 | 9052, 9007# 100083J |
| 465 | 404 | 475 | 133 | 350 | 60 | 10 | 31 | 9098 |
| 465 | 415 | 377 | 135 | 326 | 62(75) | 9/10 | 2 | 9031, 9032 |

Updated from Cheong et al 2001
ID = Identifier, 10IHW = 10th International Histocompatibility workshop, 4AOH = 4th Asia Oceania Histocompatability workshop. *9018 was heterozygous for TNFA-308
#9007 is non-57.1 at the centromeric region of the MHC from D6S1014 to HLA-DQ loci

Screening for the 57.1 Ancestral Haplotype Before Commencing Abacavir Treatment

Major histocompatibility complex region (MHC) typing was performed prospectively on all patients enrolled in the Western Australian HIV Cohort study before starting antiretroviral therapy during 2002. Patients with the full 57.1AH were not started on abacavir with the exception of one patient in whom the HLA results were not reviewed before he was inadvertently started on the drug. A second patient with HLA-B*5701 but not the other markers of the 57.1 AH C4A6 or DR7/DQ3 elected to take abacavir despite the increased risk. 47 HLA-B*5701 negative patients were started on abacavir.

Definite abacavir hypersensitivity (ABC HSR) occurred within two weeks in the two patients with HLA-B*5701. All forty-seven HLA-B*5701 negative individuals exposed to abacavir tolerated the drug giving a 95% confidence interval for the incidence of 0 to 0.75% in this population. This compares with a 9% incidence (18 out of 200) of ABC HSR in Western Australian patients [Mallal et al (2002) Lancet, 359:727-732] before the introduction of HLA screening. On the basis of these findings it is recommended that no patient with HLA-B*5701 be commenced on abacavir unless it can be demonstrated that the patient does not have the HSP 70.2 region of the 57.1AH.

Conclusion: In conclusion, finer scale haplotype analysis confirms the 57.1 AH specific linkage in ABC HSR patients and has identified a 14 kb region extending from HSP70.2 to HSP70.1 gene that confers susceptibility to ABC hypersensitivity.

TABLE 6
SNP carried on the 57.1 AH

AL662834
(COX,8.1 AH)

| nucleotide | 94085 | 93861 | 93697 | 93547 | 93532 | 93333 | 92890 | 92167 |
|---|---|---|---|---|---|---|---|---|
| | rs668558 | rs3036297 | rs5399889 | rs541340 | rs506770 | rs483638 | rs1061581 | rs562047 |

| 92059 | 92053 | 91751 | 91742 | 91037 | 81561 | 81546 | 81351 | 81338 |
|---|---|---|---|---|---|---|---|---|
| rs1043620 | rs1043619 | rs2607019 | NR | NR | rs508633 | rs508603 | rs541340 | rs506770 |

| 81157 | 81137 | 80694 | 79988 | 79971 | 79863 | 79857 | 79619 |
|---|---|---|---|---|---|---|---|
| NR | rs483638 | rs1061551 | NR | RS562047 | RS1043620 | rs1043619 | rs1043618 |

SNP name
Gene

| Location | | | Hsp70.2 | | | | | | | | | | |
|---|---|---|---|---|---|---|---|---|---|---|---|---|---|
| (strand) | | | — | — | — | — | — | — | — | — | — | | |
| | | | GC | TG | GC | AG | GA | CG | CT | GC | G | | G |
| SAMPLE ID | CA | AGTTA- | G620G | V570V | A565A | S499N | Q351Q | D110E | I74I | R72R | A | AC | A |
| 57.1 | | | C | T | G | A | A | G | C | G | | | G |
| 7.1 | C | AGTTA | C | T | G | A | G | G | T | G | A | A | G |
| 8.1 | C | AGTTA | C | T | G | A | G | G | C | G | A | A | A |
| 18.2 | A | AGTTA | G | T | G | A | G | G | C | G | A | A | A |

SNP name
Gene

| Location | Hsp70.1 | | | | | | | | | | | |
|---|---|---|---|---|---|---|---|---|---|---|---|---|
| (strand) | — | — | — | — | — | — | — | | — | — | — | — |
| | AG | CT | CA | GC | CT | AG | GA | CT | CG | CT | GC | GC |
| SAMPLE ID | V640V | P635P | V570V | A565A | N505N | N499S | Q351Q | 116 | D110E | I74I | R72R | −27 |
| 57.1 | | | | G | T | A | G | C | G | C | G | G |
| 7.1 | A | C | C | G | C | A | G | C | G | T | G | G |
| 8.1 | A | C | C | C | C | A | G | C | G | C | G | C |
| 18.2 | A | C | | C | C | A | A | C | C | C | G | C |

7.1 AH: GenBank Accession number: NT_034874
8.1 AH: GenBank Accession number: AL662834
18.2 AH: GenBank Accession number: AL929592
NR: SNP number not known

### EXAMPLE 3

Testing of SNPs to Determine the Best Test for ABC HSR

Screening of the two population studies (200 in retrospective study [Mallal et al (2002) Lancet, 359:727-732] and 49 in the prospective study) using our stored DNA on patients who were either sensitive or tolerant to abacavir, was performed to validate the test and to determine the sensitivity, specificity, positive and negative predictive values.

More specifically, the sensitivity, specificity, positive and negative predictive values of the SNP combinations were carried out as done for HLA-B*5701 and HLA-B*5701+DR7+DQ3 (the entire 57.1 haplotype) as described in Example 1 and Mallal et al (2002) Lancet, 359:727-732. The test with the highest positive and negative predictive values is the most useful and therefore the most preferred.

SNP Mining

The SNPs carried on the 57.1AH identified by sequence comparison of the 8.1, 7.1 and 18.1 Ahs were examined using pyrosequencing SNP assay.

Specific Amplification of the HSP70.2, HSP70.1 and Inter-Genic Regions

HSP70.2, HSP70.1 genes, the inter-genic region and the 5′ to 3′ regions of the two genes was specifically amplified. Nested PCR amplification with specific primers to amplify the first round followed by a second round amplification using the pyrosequencing primers was carried out.

The region around the SNP was amplified using pyrosequencing primers. Table 5 lists the primers used for SNP detection using the pyrosequencing assay as defined in Example 2.

Identification of the SNP Within the HSP70.2 and HSP70.1 Inter-Genic Region After Sequencing of the 57.1AH

SNP typing of the entire 249 patients was carried out by sequencing using the available primers and conditions as shown in Table 3. As would be understood by a person skilled in the art, alternate methods of SNP detection, including pyrosequencing assay, RFLP, SSO and SSP may also be used to detect SNPs.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention should not be unduly limited to such specific embodiments. Indeed, various modifications of the described modes for carrying out the invention, which are obvious to those skilled in molecular biology or related fields, are intended to be within the scope of the description provided.

1. A method for identifying whether a patient will exhibit a hypersensitive reaction or like-reaction to treatment with abacavir comprising the step of: typing the patient for the presence of the 57.1 ancestral haplotype.

2. A method according to claim 1 wherein the patient having the 57.1 ancestral haplotype is in a high risk category of presenting a hypersensitive reaction following the administration of abacavir.

3. A method according to claim 1 wherein the patient may be identified as possessing the 57.1 ancestral haplotype by typing the patient for one or more allele (s) or marker (s) of those alleles that are characteristic of the haplotype.

4. A method according to claim 3 wherein the common alleles comprising the 57.1 ancestral haplotype include HLA-DRB1*0701 (DR7),DQB1*0303 (DQ3),D6S1014*137, C4A6,rs419788G(SKI2W), rs437179G(SKI2W), RS494620C (NG22), GenBank Accession number AF134726 nucleotide 34569A (G9A), rs1061581A (Hsp70.2), rs562047G (Hsp70.1),rs1043618G (Hsp70.1), rs2227957C (HspHom),N3˜2˜5SnRNP*461, N3˜2˜1 MSH5*421,N3 2 2CLCP*369, D6S273*135, TNF-238A,MICA*194,MIB*344, HLA-B*5701.

5. A method according to claim 1 wherein individual alleles making up the 57.1 ancestral haplotype may be either haplospecific (eg HLA-B*5701 and 57.1 AH) or common to multiple ancestral haplotypes (eg HLA-DRB1*0701 represented in 57.1, 47.1 and 13.1 haplotypes).

6. A method according to claim 4 wherein typing will seek to identify at least a single marker that is haplospecific for the 57.1 ancestral haplotype (such as HLA-B*5701) and or a combination of two or more alleles that may be common to multiple ancestral haplotypes but when detected together in one individual are sufficiently distinctive for the 57.1 ancestral haplotype.

7. A method according to claim 1 wherein the patient is screened for markers specific to the HLA subtype HLA-B*5701.

8. A method for detecting a patient's hypersensitivity to abacavir, comprising the step of: screening the patient for at least a marker specific to HLA-B*5701 and at least a marker (s) specific for one or more of the HLA subtypes selected from the group consisting of C4A6, HLA-DRB1*0701 or HLA-DQB1*0303.

9. A method of screening a patient for HLA-B*5701 and one or more HLA subtypes selected from the group consisting of: C4A6, HLA-DRB1*0701 or HLA-DQB1*0303 wherein said screening provides a surrogate marker for the presence of the entire 57.1 ancestral haplotype.

10. A method according to claim 8 wherein the patient is screened to detect the presence of at least HLA subtype HLA-B*5701 and one or more of C4A6, HLA-DRB 1*0701 or HLA-DQB1*0303.

11. A method according to claim 8 wherein the patient is HLA typed for HLA-B*5701 and at least C4A6.

12. A method according to claim 8 wherein the patient is HLA typed for HLA-B*5701 and at least HLA-DRB1*0701.

13. A method according to claim 8 wherein the patient is HLA typed for HLA-B*5701 and at least HLA-DQB1*0303.

14. A method according to claim 8 wherein the patient is HLA typed for HLA-B*5701 and at least HLA-DQB1*0303 and HLA-DRB1*0701.

15. A method according to claim 8 wherein the patients are subtyped for the presence of: (a) HLA-B*5701, (b) HLA-B*5701, HLA-DRB1*0701 and HLA-DQB1*0303 or (c) HLA-B*5701 and HLA-DQB1*0303.

16. A method according to claim 1 wherein other markers within the 57.1 ancestral haplotype may also be detected.

17. A method according to claim 16 wherein one or more markers is specific for TNF-238A.

18. A method according to claim 1 wherein the predictive power of the test for abacavir hypersensitivity may be improved by also screening for the single nucleotide polymorphism TNF-308A.

19. A method according to claim 1 wherein C4 allotyping is also performed for the C4A6 allele.

20. A method according to claim 19 wherein allotyping is performed by immunofixation electrophoresis.

21. A method of excluding a patient from abacavir therapy, comprising performing a method according to claim 1 wherein the patient is excluded from such therapy when said patient is found to display a positive association with either the 57.1 ancestral haplotype or more preferably for the presence of HLA-B*5701 and one or more of the subtypes C4A6, HLA-DRB1*0701 or HLA-DQB1*0303.

22. (canceled)

23. (canceled)

24. A method for detecting a patient's predisposition to hypersensitivity to abacavir, comprising the steps of: screening the patient for a marker or combination of markers specific to the 57.1 ancestral haplotype in the region between 2 kb 3′ of the HSP 70.2 gene and 2 kb 5′ of the 70.1 gene Hsp70 region.

25. A method according to claim 24 wherein the patient is screened for markers specific to the Hsp70 region of the 57.1 ancestral haplotype.

26. A method according to claim 24 wherein the patient may be identified as possessing the 57.1 ancestral haplotype by typing the patient for one or more allele (s) or marker (s) of those alleles that are characteristic of the Hsp70 region of the 57.1 ancestral haplotype.

27. A method for detecting a patient's predisposition to abacavir hypersensitivity, comprising the step of: screening the patient for at least a marker specific to HLA-B*5701 and at least a marker (s) specific for one or more of the Hsp70 region.

28. A kit containing components for determining a patient's predisposition to abacavir hypersensitivity.
